(19) United States
(12) Patent Application Publication
(10) Pub. No.: US 2004/0236290 A1
(43) Pub. Date: Nov. 25, 2004

(54) Minimally invasive vascular apparatus modified to minimize scarring at introduction site

(76) Inventor: Stephan A. Zimmermann, Healdsburg, CA (US)

Correspondence Address: MEDTRONIC VASCULAR, INC., IP LEGAL DEPARTMENT, 3576 UNOCAL PLACE, SANTA ROSA, CA, 95403, US

(21) Appl. No.: 10/830,903

(22) Filed: Apr. 22, 2004

Related U.S. Application Data
(60) Provisional application No. 60/465,178, filed on Apr. 23, 2003.

(51) Int. Cl. A61M 25/00

(52) U.S. Cl. 604/265

(57) ABSTRACT

In one embodiment, a medical device for insertion into a body is disclosed and includes a device body having a proximal section defining an inlet port and a distal section having a delivery port located thereon and at least one anti-inflammatory coating selectively applied to the device body, the anti-inflammatory coating configured to minimize inflammation of tissue in contact therewith.

### CROSS REFERENCE TO RELATED APPLICATIONS

The present application claims priority to U.S. Provisional Application Serial No. 60/465,178 filed Apr. 23, 2003, the entire contents of which are herein incorporated by references in their entirety.

### BACKGROUND OF THE INVENTION

A variety of minimally invasive medical and surgical procedures require the insertion of various instruments, such as guidewires, needles, cannulas, and catheters, into an artery, vein, or other vascular structure of a patient. During these procedures, a needle or other puncture device is inserted through the skin of a patient into an underlying blood vessel or other vascular structure, thereby forming a insertion site therein and permitting access to the internal passageway of the vascular structure. Thereafter, an instrument such as a cannula is inserted into the insertion site and advanced through the circulatory system to an area of interest. Ideally, a single insertion site will be used for multiple minimally invasive surgical procedures over a period of time, thereby decreasing patient discomfort while limiting the amount of scar tissue formed on the blood vessel wall and skin of the patient.

Generally, it is preferable that the puncture hole formed in the skin and blood vessel be as small as possible thereby reducing the amount of leakage around the surgical instrument and minimizing the subsequent wound size. Large puncture holes may result in an increased likelihood of post operative complications such as infection and the formation of large areas of scar tissue formed on the blood vessel wall and the skin surface proximate to the insertion site. As a result, the subsequent introduction of instruments through these to the insertion site. As a result, the subsequent introduction of instruments through these large areas of scar tissue has often proven problematic. For example, the advancement of a needle or puncture device through scar tissue is more difficult than non-scar tissue. In addition, effective wound closure is often more difficult when the puncture hole is formed in scar tissue and may require the physician to perform a more time consuming wound closing procedure.

Thus, in light of the foregoing, there is an ongoing need for a minimally invasive apparatus modified to minimize tissue scarring.

### BRIEF SUMMARY OF THE INVENTION

The medical device disclosed herein has been modified to reduce or eliminate tissue inflammation or scarring proximate to an insertions site. More particularly, the modified device include at least one anti-inflammatory or scar-reducing coating thereon. In one embodiment, the anti-inflammatory or scar-reducing coating is capable reducing or eliminating inflammation or scarring at an insertion site, thereby permitting multiple minimally invasive procedures to be performed using a single insertion point formed within the skin of a patient.

In one embodiment, a medical device for insertion into a body is disclosed and includes a device body having a proximal section defining an inlet port and a distal section having a delivery port located thereon and at least one anti-inflammatory coating selectively applied to the device body, the anti-inflammatory coating configured to minimize inflammation of tissue in contact therewith.

In another embodiment, a medical device for insertion into a body is disclosed and includes a device body having a proximal section defining an inlet port and a distal section having a delivery port located thereon, a base coating applied to the device body, and at least one anti-inflammatory coating applied to the device body over the base coating, the anti-inflammatory coating configured to minimize inflammation of tissue in contact therewith.

In another embodiment, a medical device for insertion into a body is disclosed and includes a device body having a proximal section defining an inlet port and a distal section having a delivery port located thereon, a base coating applied to the device body, at least one anti-inflammatory coating applied to the device body over the base coating, the anti-inflammatory coating configured to minimize inflammation of tissue in contact therewith, and at least one therapeutic coating applied to the device body over the at least one anti-inflammatory coating.

### BRIEF DESCRIPTION OF THE DRAWINGS

An embodiment of the minimally invasive vascular apparatus modified to minimize scarring at introduction site will be explained in more detail by way of the accompanying drawings, wherein:

FIG. 1 shows a perspective view of an embodiment of a medical device having an anti-inflammatory coating applied thereto;

FIG. 2 shows a cross-sectional view of a medical device of FIG. 1 having an anti-inflammatory coating applied thereto;

FIG. 3 shows a cross-sectional view of another embodiment of a medical device of FIG. 1 having an anti-inflammatory coating applied thereto;

FIG. 4 shows a cross-sectional view of another embodiment of a medical device of FIG. 1 having an anti-inflammatory coating applied thereto;

FIG. 5 shows perspective view of an embodiment of a medical device of FIG. 1 being inserted into tissue; and

FIG. 6 shows perspective view of a guidewire inserted through an embodiment of a medical device of FIG. 5 and positioned within vessel within the tissue.

### DETAILED DESCRIPTION OF THE INVENTION

The present application discloses a variety of medical devices modified to reduce or eliminate inflammation or scarring during or following the insertion thereof into tissue of a patient. More particularly, the medical device has been coated with at least one biologically compatible anti-inflammatory and/or scar-reducing coating configured to reduce or eliminate inflammation and/or scarring of tissue positioned proximate thereto. In one embodiment, the coatings are applied to minimally invasive surgical implements, including, for example, trocars, catheters including indwelling catheters, introducers, sheaths, needles, retainers, retractors, guidewires, obturators, stylets, sheaths, shunts, staples, screws, tissue fasteners, mounts, biosensors, peritoneal plugs, extracorporeal blood circuit devices, and cannulas. In another embodiment, the coatings are applied to medical devices used during conventional surgical procedures, such as retractors, bone anchors, and pins.

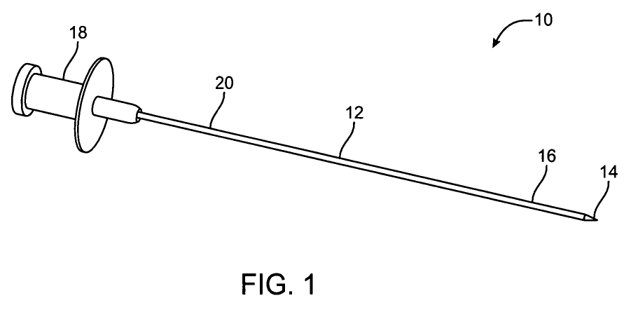

FIG. 1 shows an embodiment of a modified medical device. In the illustrated embodiment, the medical device 10 includes a device body 12 defining at least one internal lumen (not shown). A delivery port 14 in communication with the internal lumen (not shown) may be located at the distal section 16 of the device body 12. In the illustrated embodiment, the distal section 16 of the device body 12 is configured to puncture or be inserted through tissue. In an alternate embodiment, the distal section 16 may be atraumatic. An inlet body 18 is positioned on the proximal section 20 of the device body 12 and in communication with delivery port 14 through the at least one internal lumen (not shown) formed within the device body 12. The medical device 10 may be manufactured from a variety of materials, including, without limitation, biologically-compatible metals such as stainless steel, titanium, nickel-titanium alloys, and tantalum; biologically compatible polymers such as poly(L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D, L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes, biomolecules, fibrin, fibrinogen, cellulose, starch, collagen, hyaluronic acid, polyurethanes, silicones, polyesters, polyolefins, polyisobutylene, ethylene-alphaolefin copolymers, acrylic polymers, acrylic copolymers, ethylene-covinylacetate, polybutylmethacrylate, vinyl halide polymers, vinyl halide copolymers, polyvinyl chloride, polyvinyl ethers, polyvinyl methyl ether, polyvinylidene halides, polyvinylidene fluoride, polyvinylidene chloride, polyacrylonitrile, polyvinyl ketones, polyvinyl aromatics, polystyrene, polyvinyl esters, polyvinyl acetate, copolymers of vinyl monomers, ethylene-methyl methacrylate copolymers, acrylonitrile-styrene copolymers, ABS resins, ethylene-vinyl acetate copolymers, polyamides, Nylon 66, polycaprolactam, alkyd resins, polycarbonates, polyoxymethylenes, polyimides, polyethers, epoxy resins, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose.

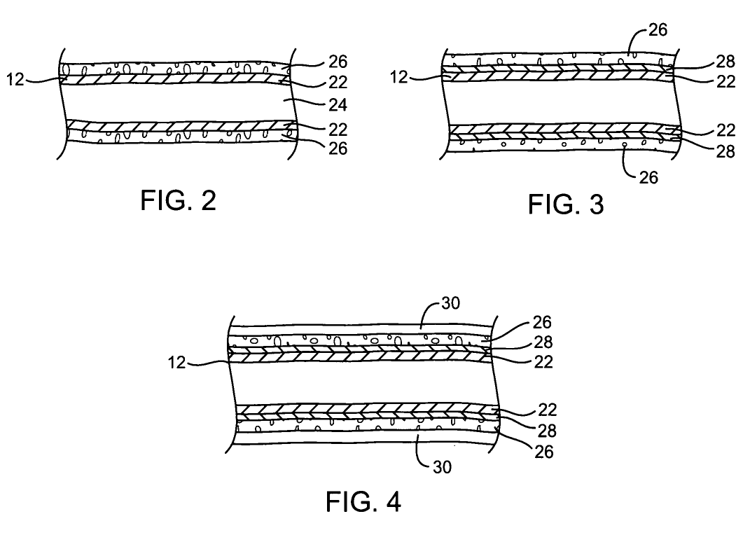

FIGS. 2-4 show various cross sectional views of embodiment of the medical device 10. As shown in FIG. 2, the device body 12 includes an outer wall 22 defining at least one internal lumen 24. In the illustrated embodiment, at least one anti-inflammatory or scar-reducing coating 26 may be selectively applied to the outer wall 22 of the device body 12. In one embodiment, the length of the device body 12 may be coated with the anti-inflammatory or scar-reducing agent 26. Alternatively, a section of the device body 12 may be selectively coated with the anti-inflammatory or scar-reducing agent 26, while another section of the device body 12 is coated with at least other material (not shown). For example, the proximal section 22 of the device body 12 may be coated with the anti-inflammatory or scar-reducing agent while the distal section 16 is coated with a lubricious or therapeutic coating (not shown). FIG. 3 show another embodiment wherein the outer wall 22 of the device body 12 includes at least one base coating 28 configured to receive at least one anti-inflammatory of scar-reducing coating 26 thereon. Exemplary base coatings 28 may include paralene and/or a corrosion resistance materials or treatments. FIG. 4 shows another embodiment of the device body 12 wherein at least one therapeutic agent 30 has been applied to the outer wall 22 over the anti-inflammatory or scar-reducing agent 26. Those skilled in the art will appreciate that the therapeutic agent 30 may be applied prior to, concurrent with, or following the application of the anti-inflammatory or scar-reducing agent 26. Exemplary therapeutic agents 30 include, without limitation, anti-coagulants, coagulants, antirestenotic compounds or agents, lubricious coatings, marker materials or agents, polytetrafluoroethylene, anti-thrombotic agents, platlet-derived growth factor (PDGF), tranforming growth factor-beta (TGF-beta), heparin, anti-inflamatory agents, anti-proliferation agents, rapamycin, angiopeptin, methotrexate, paclitaxel, anti-microbial agents, anti-metabolic agents, anti-platlet agents, Nitric Oxide releasing agents, chaperone inhibitors, geldanamycin, glitazones, metalloproteinase inhibitors (MMPI), antisense polynucleotides, and transforming nucleotides.

Exemplary anti-inflammatory or scar-reducing agents 26 may include, without limitation, steroidal anti-inflammatories (SAIDs), non-steroidal anti-inflammatories (NSAIDs), fibroblast growth factors (FGF), collagen synthesis inhibitors, fibrinectin inhibitors, superoxide dismutuse (SOD), non-methylene interrupted fatty acids (NMIFAs) 5,11,14,20:3, as well as other materials capable of reducing or eliminating tissue inflammation and tissue scarring. In an alternate embodiment, the anti-inflammatory or scar-reducing coating 26 may include multiple therapeutic agents configured to reduce or eliminate tissue inflammation and scarring. For example, the anti-inflammatory or scar-reducing coating 26 may include anti-restenotic materials or lubricious materials configured to reduce or eliminate inflammation to the interior of a vessel wall. The anti-inflammatory or scar-reducing agent 26 may be applied to the medical device 10 in a variety of ways, including, for example, sprayed, dipped, and vapor deposited.

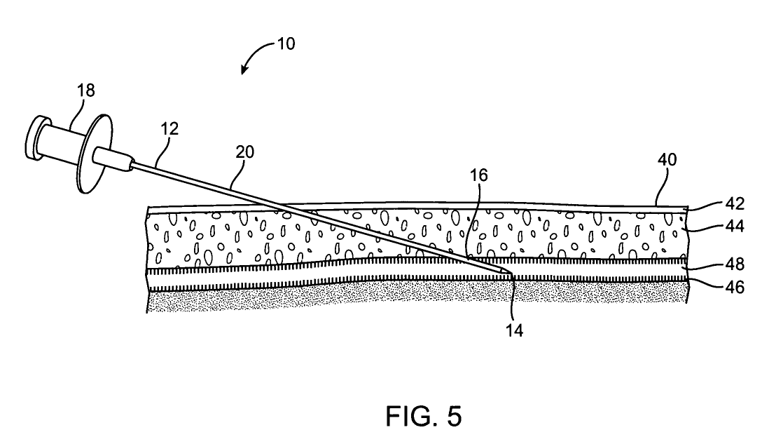

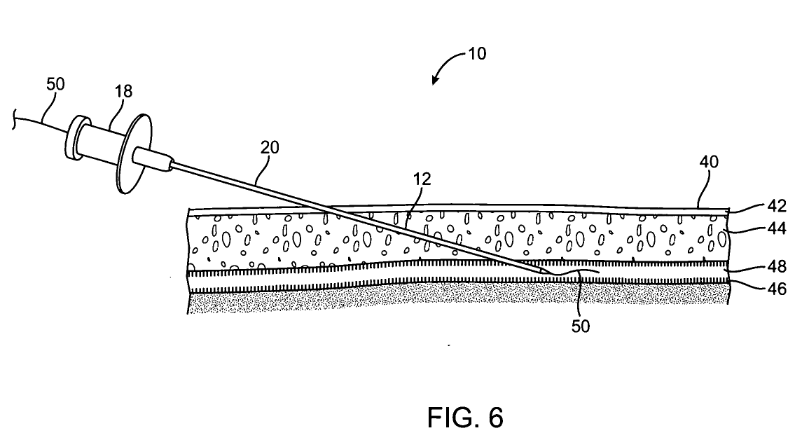

FIGS. 5 and 6 show an embodiment of the medical device 10 during use. As shown, the delivery port 14 located on the distal section 16 of the device body 12 is inserted into the tissue 40 and advanced through an outer layer 42 and intermediate layer 44 of the tissue 40, and arriving at a vessel 46. Thereafter, the distal section 16 is advanced through the outer wall of the vessel 46 and communicates with the interior portion 48 thereof. As a result, the inlet body 18 located on the proximal section 20 of the device body 12 may communicate with the interior portion 48 of the vessel 46. Thereafter, a minimally invasive surgical device or guidewire 50 may delivered to the interior portion 48 of the vessel 46 through the medical device 10. As shown, the guidewire 50 is inserted into the inlet body 18 and advanced through the internal lumen 24 (see FIG. 2) formed in the device body 12. The guidewire 50 exits the device body 12 through the delivery port 14 positioned within the interior portion 48 of the vessel 46. As shown in FIGS. 5 and 6, the length of the device body 12 may include at least one anti-inflammatory or scar-reducing coating, thereby reducing or eliminating inflammation and scarring of the outer layer 42, the intermediate layer 44, as well as the vessel 46. In an alternate embodiment, the at least one anti-inflammatory or scar-reducing coating may be selectively applied to the device body 12.

In closing it is understood that the embodiments disclosed herein are illustrative of the principles of the invention and are not intended to limit the scope of the invention. For example, at least one anti-inflammatory or scar-reducing coating may be applied to a variety of medical devices, including, without limitation, minimally invasive surgical implements, trocars, catheters, indwelling catheters, introducers, sheaths, needles, retainers, retractors, guidewires, obturators, stylets, sheaths, shunts, staples, screws, tissue fasteners, mounts, biosensors, peritoneal plugs, extracorporeal blood circuit devices, cannulas, retractors, bone anchors, and pins.

What is claimed is:

1. A medical device for insertion into a body, comprising;
a device body having a proximal section defining an inlet port and a distal section having a delivery port located thereon; and
at least one anti-inflammatory coating selectively applied to the device body, the anti-inflammatory coating configured to minimize inflammation of tissue in contact therewith.

2. The device of claim 1 wherein the medical device is selected from the group consisting of minimally invasive surgical implements, trocars, catheters, indwelling catheters, introducers, sheaths, needles, retainers, retractors, guidewires, obturators, stylets, sheaths, shunts, staples, screws, tissue fasteners, mounts, biosensors, peritoneal plugs, extracorporeal blood circuit devices, cannulas, retractors, bone anchors, and pins.

3. The device of claim 1 wherein the anti-inflammatory coating is selectively applied to the proximal section of the device body.

4. The device of claim 1 wherein the anti-inflammatory coating is selectively applied to the distal section of the device body.

5. The device of claim 1 wherein the anti-inflammatory coating is applied to the proximal and distal section of the device body.

6. The device of claim 1 wherein the anti-inflammatory coating is selected from the group consisting of anti-inflammatory or scar-reducing agents 26 may include, without limitation, steroidal anti-inflammatories (SAIDs), non-steroidal anti-inflammatories (NSAIDs), fibroblast growth factors (FGF), collagen synthesis inhibitors, fibrinectin inhibitors, superoxide dismutuse (SOD), non-methylene interrupted fatty acids (NMIFAs) 5,11,14,20:3.

7. The device of claim 1 wherein the anti-inflammatory coating is lubricious.

8. The device of claim 1 further comprising a base coating applied to the device body and configured to receive at least one anti-inflammatory coating thereon.

9. The device of claim 8 wherein the base coating comprises paralene.

10. The device of claim 1 further comprising at least one therapeutic coating applied thereto.

11. The device of claim 10 wherein the therapeutic coating is applied over the anti-inflammatory coating.

12. The device of claim 10 wherein the therapeutic coating is applied under the anti-inflammatory coating.

13. The device of claim 10 wherein the therapeutic coating is lubricious.

14. The device of claim 10 wherein the therapeutic agent is selected from the group consisting of, anti-coagulants, coagulants, anti-restenotic compounds or agents, lubricious coatings, marker materials or agents, polytetrafluoroethylene, anti-thrombotic agents, platlet-derived growth factor (PDGF), tranforming growth factor-beta (TGF-beta), heparin, anti-inflamatory agents, anti-proliferation agents, rapamycln, angiopeptin, methotrexate, paclitaxel, anti-microbial agents, anti-metabolic agents, anti-platlet agents, Nitric Oxide releasing agents, chaperone inhibitors, geldanamycin, glitazones, metalloproteinase inhibitors (MMPI), antisense polynucleotides, and transforming nucleotides.

15. A medical device for insertion into a body, comprising;
a device body having a proximal section defining an inlet port and a distal section having a delivery port located thereon;
a base coating applied to the device body; and
at least one anti-inflammatory coating applied over the base coating, the anti-inflammatory coating configured to minimize inflammation of tissue in contact therewith.

16. The device of claim 15 wherein the base coating comprises paralene.

17. A medical device for insertion into a body, comprising;
a device body having a proximal section defining an inlet port and a distal section having a delivery port located thereon;
a base coating applied to the device body;
at least one anti-inflammatory coating applied to the device body over the base coating, the anti-inflammatory coating configured to minimize inflammation of tissue in contact therewith; and
at least one therapeutic coating applied to the device body over the at least one anti-inflammatory coating.
